(19) United States
(12) Patent Application Publication
(10) Pub. No.: US 2024/0420842 A1
(43) Pub. Date: Dec. 19, 2024

(54) COMPUTER-IMPLEMENTED METHOD AND SYSTEM FOR DIAGNOSING DISEASES THAT AFFECT MORPHOLOGICAL CHARACTERISTICS AND THE CYTOPLASMIC COMPLEXITY OF BLOOD CELLS

(71) Applicant: RobotDreams GmbH, Graz (AT)

(72) Inventors: Aleksandr PUSHKIN, Saint-Petersburg (RU); Dimitrij SHULKIN, Norderstedt (DE); Sergey CHERTKOV, Hamburg (DE)

(21) Appl. No.: 18/626,979

(22) Filed: Sep. 4, 2024

Related U.S. Application Data
(63) Continuation of application No. PCT/EP2022/077297, filed on Sep. 30, 2022.

(30) Foreign Application Priority Data
Oct. 5, 2021 (DE) 10 2021 004 988.8

(51) Int. Cl. G16H 50/20; G16H 10/40; G16H 50/70

(52) U.S. Cl. CPC G16H 50/20; G16H 10/40; G16H 50/70

(57) ABSTRACT

A computer-implemented method for diagnosing diseases that affect the morphological characteristics and the cytoplasmic complexity of blood cells, the method comprising: a) obtaining blood parameters from at least one patient, the blood parameters comprising at least two measured properties of individual cells from full blood analysis, the individual cells comprising leukocytes, the measured properties of the leukocytes comprising the size and granularity of the cytoplasm and the structure of the cell nucleus; b) creating at least one scatter plot, each axis of the scatter plot comprising a different measured property of the individual cells; c) ascertaining at least one cluster in at least one scatter plot, the clusters comprising the subpopulations of leukocytes, the subpopulations comprising monocytes, lymphocytes, basophils, neutrophils and eosinophils; d) combining the property elements of the ascertained clusters to form a 1-dimensional global vector, the arrangement of the property elements in the global vector comprising an arrangement by associated cluster; e) reducing the dimension of the global vector; f) diagnosing at least one disease by means of an ensemble, the ensemble comprising at least one machine learning model and at least one deep learning model, the at least one machine learning model receiving at least one reduced global vector as an input variable and the at least one deep learning model receiving at least one scatter plot image as an input variable; and g) automatically generating a report which comprises at least one result of the diagnosis of at least one disease.

### TECHNICAL FIELD

The invention relates to clinical laboratory diagnostics and describes a computer-implemented method for diagnosing diseases that affect the morphological characteristics and the cytoplasmic complexity of blood cells.

### TECHNICAL BACKGROUND

Clinical blood analysis is one of the most accessible diagnostic methods in practical medicine as it reflects systemic pathological processes in the human body on the basis of a quantitative evaluation of the cell composition and blood morphology. Circulating blood cells continuously permeate almost all tissues in the human body at a high flow rate, and their states of maturation, activation, proliferation and ageing reflect the current pathophysiological conditions: health, acute response to pathology, chronic compensation for diseases and finally decompensation.

The routine measurement of the complete blood count comprises the measurement of individual-cell characteristics in tens of thousands of blood cells and provides a comprehensive overview of these pathophysiological conditions. In each routine blood count, high-dimensional individual-cell information of white blood cells (leukocytes) is measured. In the process, in the hematological analyzer, various morphological characteristics such as size, complexity, lobularity and granularity are measured, which make it possible to differentiate the five subpopulations of mature leukocytes into neutrophils, lymphocytes, monocytes, eosinophils and basophils (Takubo T, Tatsumi N. Further evolution and leukocyte differential using an automated blood cell counter. Rinsho Byori. 1995 September; 43(9):925-30. Japanese. PMID: 7474456.).

Clinical decisions are currently based only on a few derived statistics. Due to the large amount of information which is obtained through the patient's blood count, physicians are often not able to interpret it completely in relation to the clinical situation. (Chaudhury A, Noiret L, Higgins J M. White blood cell population dynamics for risk stratification of acute coronary syndrome. Proc Natl Acad Sci USA. 2017 Nov. 14; 114(46):12344-12349. doi: 10.1073/pnas.1709228114. Epub 2017 Oct. 27. PMID: 29087321; PMCID: PMC5699055.).

The enormous potential of the complete blood count was recognized, however, wherein earlier attempts were made to detect infections at an early stage through the identification of immature granulocytes or to determine the prognosis for some malignant diseases by counting the number of (leukocytes) with atypical characteristics (Statland B E, Winkel P, Harris S C, Burdsall M J, Saunders A M. Evaluation of biologic sources of variation of leukocyte counts and other hematologic quantities using very precise automated analyzers. Am J Clin Pathol. 1978 January; 69(1):48-54. doi: 10.1093/ajcp/69.1.48. PMID: 563672.).

Although these endeavors had only limited success, they indicate the potential for an improved clinical decision aid (Gijsberts C M, den Ruijter H M, de Kleijn D P V, Huisman A, Ten Berg M J, van Wijk R H A, Asselbergs F W, Voskuil M, Pasterkamp G, van Solinge W W, Hoefer I E. Hematological Parameters Improve Prediction of Mortality and Secondary Adverse Events in Coronary Angiography Patients: A Longitudinal Cohort Study. Medicine (Baltimore). 2015 November; 94(45):e1992. doi: 10.1097/MD.0000000000001992. PMID: 26559287; PMCID: PMC4912281.).

The methods for diagnosing acute coronary syndrome (ACS) through the analysis of leukocytes using statistical-mathematical approaches were investigated in the following paper (Chaudhury A., Noiret L., Higgins J. White blood cell population dynamics for risk stratification of acute coronary syndrome. Proceedings of the National Academy of Sciences, 2017; 114(46), pp. 12344-12349).

As the amount of digital data provided by analysis systems increases, the potential for the application of machine learning methods from the field of artificial intelligence (AI) grows, in order to increase the effectiveness of the available diagnostic information in the interests of the patient and of medical institutions (Píeszko K., Hiczkiewicz J., Budzianowski P., Rzeźniczak J., Budzianowski J., Błaszczyński J., Słowiński R., Burchardt, P. Machine-Iearned models using hematological inflammation markers in the prediction of short-term acute coronary syndrome outcomes. Journal of Translational Medicine, 2018; 16(1):334).

### SUMMARY OF THE INVENTION

The object can be regarded as proposing an alternative method for diagnosing diseases, in particular diseases that affect the morphological characteristics and the cytoplasmic complexity of blood cells, in order to improve the quality of medical care of patients.

The object is achieved by a computer-implemented method described herein. Advantageous embodiments and developments will be set forth in the following description.

A computer-implemented method for diagnosing diseases that affect the morphological characteristics and the cytoplasmic complexity of blood cells is proposed, wherein the method comprises: a) obtaining blood parameters from at least one patient, wherein the blood parameters comprise at least two measured properties of the individual cells of the full blood count, wherein the individual cells comprise the leukocytes, wherein the measured properties of the leukocytes comprise the size, granularity of the cytoplasm and the structure of the cell nucleus; b) creating at least one scatter plot, wherein each axis of the scatter plot comprises a different measured property of the individual cells; c) ascertaining at least one cluster in at least one scatter plot, wherein the clusters comprise the subpopulations of the leukocytes, wherein the subpopulations comprise monocytes, lymphocytes, basophils, neutrophils and eosinophils; d) combining the property elements of the ascertained clusters to form a one-dimensional global vector, wherein the arrangement of the property elements in the global vector comprises an arrangement according to the associated clusters; e) reducing the dimension of the global vector; f) diagnosing at least one disease using an ensemble, wherein the ensemble comprises at least one machine learning model and at least one deep learning model, wherein the at least one machine learning model receives at least one reduced global vector as input variable and the at least one deep learning model receives at least one scatter plot image as input variable; and g) automatically generating a report which comprises at least one result of the diagnosis of at least one disease.

The performance of a method according to the invention is explained further below.

The patient enters the emergency department of the medical organization. Acute coronary syndrome (ACS), which affects the morphological characteristics and the cytoplasmic complexity of blood cells, is suspected. The first venous whole blood is taken from a cubital vein e.g. with the aid of a 4 ml vacuum system for taking blood samples into an e.g. Vacutest test tube (KIMA, Italy) and deposited on the inner surface of the e.g. 7.2 mg K3EDTA test tube walls. This sample is then used to diagnose diseases using the method according to the invention. Taking the sample is not part of the method.

After taking the blood sample, the test tube is stirred by turning it upside down and rotating it horizontally and vertically for 30 seconds. After that, the clinical blood examination is carried out on the automatic hematology analyzer e.g. CELL-DYN Sapphire (Abbott Laboratories, USA) in open mode. In the process, the individual cells of the total blood count are measured in a high-dimensional manner, wherein the measurements comprise the properties of the individual cells, as represented by way of example in FIG. 1.

The measurements are copied by the analyzer e.g. as FCS files or in another format and transferred to an accessible PC or mobile computing device or a cloud for machine processing. These measurements contain blood parameters as properties of leukocytes, wherein leukocytes comprise neutrophils, eosinophils, basophils, lymphocytes and monocytes, wherein the properties comprise the size, granularity, lobularity and complexity (FIG. 2).

The machine processing consists of the automatic differentiation of at least three subpopulations (neutrophils, lymphocytes, monocytes) by plotting the measured properties against each other in at least one scatter plot, as can be seen for example in FIG. 3: size against complexity and/or size against lobularity. Abbott Cell-DYN Sapphire ascertains the size of the cells by measuring the axial light loss (ALL) for each cell, the cytoplasmic and nuclear complexity by measuring the intermediate angle scatter (IAS) intensity and the lobularity by measuring the polarized side scatter (PSS) intensity.

The ascertaining of the subpopulations differentiated by their properties is effected automatically through hierarchical cluster analysis, wherein by hierarchical cluster analysis is meant a method for finding similarity structures in data sets, wherein clusters here consist of measured variables which have a smaller distance from each other than from the measured variables of other clusters (FIG. 3). In addition to hierarchical cluster analysis, the following cluster algorithms can also be used: k-means, k-medians, affinity propagation, mean shift, spectral clustering, Ward's hierarchical clustering, DBSCAN, OPTICS, Gaussian mixtures and Birch.

The elements of at least three subpopulations are then sorted and combined in a global vector:

$${V}_{GLOB} = {( \begin{matrix}\{ {V}_{Neutrophils} \} \\ \{ {V}_{Lymphocytes} \} \\ \{ {V}_{Monocytes} \}\end{matrix} )}_{n \times 1}$$

wherein the global vector comprises the vectors of at least three subpopulations differentiated by their properties:

$${V}_{Lymphocytes} = \{ \begin{matrix}{Lymphocyte}_{{Size}_{1}} \\ ⋮ \\ {Lymphocyte}_{{Size}_{n}} \\ {Lymphocyte}_{{Complexity}_{1}} \\ ⋮ \\ {Lymphocyte}_{{Complexity}_{n}}\end{matrix} \} , {V}_{Monocyte} = \{ \begin{matrix}{Monocyte}_{{Size}_{1}} \\ ⋮ \\ {Monocyte}_{{Size}_{n}} \\ {Monocyte}_{{Complexity}_{1}} \\ ⋮ \\ {Monocyte}_{{Complexity}_{n}}\end{matrix} \} \\ {V}_{Neutrophils} = \{ \begin{matrix}{Neutrophils}_{{Size}_{1}} \\ ⋮ \\ {Neutrophils}_{{Size}_{n}} \\ {Neutrophils}_{{Complexity}_{1}} \\ ⋮ \\ {Neutrophils}_{{Complexity}_{n}}\end{matrix} \}$$

wherein the lymphocyte vector comprises at least two properties (size and complexity), the monocyte vector comprises at least two properties (size and complexity) and the neutrophil vector comprises at least two properties (size and complexity). Of course, one or more further properties can also be taken into consideration here, for example the lobularity, with the result that the neutrophil vector could read as follows:

$${V}_{Neutrophils} = \{ \begin{matrix}{Neutrophils}_{{Size}_{1}} \\ ⋮ \\ {Neutrophils}_{{Size}_{n}} \\ {Neutrophils}_{{Lobularity}_{1}} \\ ⋮ \\ {Neutrophils}_{{Lobularity}_{n}} \\ {Neutrophils}_{{Complexity}_{1}} \\ ⋮ \\ {Neutrophils}_{{Complexity}_{n}}\end{matrix} \} \\ {V}_{GLOB}^{STAND} = \frac{\{ {V}_{GLOB} \} - {\{ u \}}_{Database}}{{\{ s \}}_{Database}}$$

The global vector with the property elements can then be standardized by rescaling the values in a range of [0,1] or [−1,1] or rescaling the data such that they have a mean of 0 and a standard deviation of 1 (unit variance):

$${V}_{GLOB}^{STAND} = \frac{\{ {V}_{GLOB} \} - {\{ u \}}_{Database}}{{\{ s \}}_{Database}}$$

wherein the vector {u} represents the means of the property elements of the global
vectors in the pre-built database:

$${\{ u \}}_{Database} = Mean ( {\{ {V}_{GLOB} \}}_{1}^{Patient} , … , {\{ {V}_{GLOB} \}}_{m}^{Patient} )$$

and the vector {s} represents the standard deviations of the elements of the global vectors in the pre-built database:

$${\{ s \}}_{Database} = Standard deviation ( {\{ {V}_{GLOB} \}}_{1}^{Patient} , … , {\{ {V}_{GLOB} \}}_{m}^{Patient}$$

After the standardization, a principal component analysis (PCA) is applied. The PCA can also be applied before the standardization or instead of the standardization. The aim of the PCA is to reduce the dimension of the characteristics of the standardized global vector and at the same time to retain the same amount of variability (information) of the characteristics (Dunteman G. H. Principal Component Analysis, 1989). The large size of the vectors can cause problems for machine learning as prediction models based on these data run the risk of being retrained (Kabari L. G., Nwame B. B., Principal Component Analysis (PCA)—An Effective Tool in Machine Learning, International Journals of Advanced Research in Computer Science and Software Engineering, ISSN: 2277-128X (Volume 9, Issue 5)). In addition, many of the predictors may be redundant or highly correlated, which can also result in a decrease in the diagnostic accuracy. Moreover, the PCA also increases the computational speed considerably. In addition to PCA, sparse principal component analysis, kernel principal component analysis, truncated singular value decomposition, independent component analysis, non-negative matrix factorization and latent Dirichlet allocation can also be used.

After the PCA, the number of elements in the global vector is reduced from n to the number of principal components p:

$${V}_{GLOB}^{STAND} = {( \begin{matrix}\{ {V}_{Neutrophils} \} \\ \{ {V}_{Lymphocytes} \} \\ \{ {V}_{Monocytes} \}\end{matrix} )}_{n \times 1} \to {V}_{GLOB}^{STAND \& RED} = {\{ \begin{matrix}{x}_{1} \\ ⋮ \\ {x}_{p}\end{matrix} \}}_{p \times 1} , wherein ( p \leq n ) ,$$

wherein the elements x₁ . . . xₚ of the reduced vector represent the principal components. The number p of principal components is less than or equal to the number n of elements of the global vector before the dimension reduction, i.e. p n. After the PCA, the reduced global principal component vector is used as input vector for at least one machine learning model, such as is shown for instance in FIG. 4.

Machine learning is a subfield of artificial intelligence and an umbrella term for the “artificial” generation of knowledge from experience: an artificial system learns from examples and can generalize these after completion of the learning phase. For this, the machine learning algorithms build a statistical model on the basis of training data. This means that the examples introduced in the learning phase are not simply committed to memory, but patterns and regularities in the learning data are recognized. In this way, the system can also evaluate unknown data (learning transfer).

If at least two (machine) learning models are used for the prediction, the term ensemble is used. Ensembles are a paradigm of machine learning in which several models are trained in order to solve the same problem and to combine in order to achieve better results (Langley P., Elements of Machine Learning, 1996), wherein an ensemble can contain artificial neural networks and/or k-nearest neighbors models and/or random forest models and/or decision trees and/or AdaBoost models and/or gradient boosting machines (GBMs) and/or bootstrap aggregation models and/or stacked generalization models and/or logistic regression models and/or Bayesian models and/or support vector machines (SVMs).

Whereas the global vector, such as is shown for instance in FIG. 4, is used as input variable for at least one machine learning model, at least one global scatter plot is used as input image in the form of an input matrix for at least one deep learning model, such as is shown for instance in FIG. 5, wherein a global scatter plot consists of at least one scatter plot, wherein the deep learning models could comprise convolutional neural networks (CNNs).

In the area of deep learning, a CNN is a class of artificial neural network, which is used most frequently for the analysis of visual images (Valueva, M. V.; Nagornov, N. N.; Lyakhov, P. A.; Valuev, G. V.; Chervyakov, N. I. (2020). “Application of the residue number system to reduce hardware costs of the convolutional neural network implementation”. Mathematics and Computers in Simulation. Elsevier BV. 177: 232-243. doi:10.1016/j.matcom.2020.04.031. ISSN 0378-4754.). A CNN substantially consists of filters (convolutional layer) and aggregation layers (pooling layer), which repeat in an alternating manner, and at the end of one or more layers of “normal” fully connected neurons (dense/fully connected layer). A CNN, with its filters, recognizes structures in the images independently of location. The type of filter is not predefined, but rather learned by the neural network. With each filter level, the abstraction level of the network increases. What abstractions ultimately result in the activation of the posterior layer follows from the characteristic features of the predefined classes which are to be detected.

Scatter plots are created by plotting the measured properties of the individual cells of the leukocytes against each other, such as is represented by way of example in FIG. 6: size against complexity, size against granularity, size against lobularity, complexity against granularity, complexity against lobularity, granularity against lobularity. It is conceivable that—depending on the analyzer—fewer properties are also measured, for instance only the three properties cell size, cell complexity and cell contents. It is assumed that virtually all analyzers measure at least two properties, i.e. the cell size and complexity, and, in the simplest scatter plots, at least these two properties are plotted against each other. The individual scatter plots in FIG. 6 can be added to the global scatter plot, wherein the order of the individual scatter plots in the global scatter plot can be changed.

In the method, either the diagnosis of a disease is characterized in that the votes from several trained machine and deep learning models are added together (hard voting), wherein the diagnosis of a disease with the most votes is chosen, as represented for example in FIG. 7.

Or, in the method, the determination of the diagnosis of a disease is characterized in that the probabilities from several trained machine and deep learning models are added together (soft voting), wherein the diagnosis of a disease is output from the weighted mean of the greatest cumulative probability for each ensemble, as shown for example in FIG. 8.

The machine and deep learning models are trained on the basis of a pre-built database, such as is shown by way of example in FIG. 9, wherein the database consists of the measured blood parameters. The database is subdivided into two datasets Xₜᵣₐᵢₙ and Xₜₑₛₜ, wherein Xₜᵣₐᵢₙ is used for the training of the models and Xₜₑₛₜ is used for the evaluation of the quality of the models.

For the training and evaluation of models which require global vectors as input variable, these are derived from the datasets according to the above-described approaches:

$$[ {X}_{train} , {X}_{test} ] = {[ {\{ {V}_{GLOB} \}}_{n \times 1}^{{1}^{st} patient} , {\{ {V}_{GLOB} \}}_{n \times 1}^{{2}^{nd} patient} , … , {\{ {V}_{GLOB} \}}_{n \times 1}^{{m}^{th} patient} ]}_{n \times m}$$

wherein n represents the total number of property elements and m represents the number of patients in the datasets. The datasets are standardized according to the above-named approach and then reduced according to the PCA approach:

$$[ {X}_{train} , {X}_{test} ] = {[ {\{ {V}_{GLOB}^{STAND \& RED} \}}_{p \times 1}^{{1}^{st} patient} , {\{ {V}_{GLOB}^{STAND \& RED} \}}_{p \times 1}^{{2}^{nd} patient} , \\ … , {\{ {V}_{GLOB}^{STAND \& RED} \}}_{p \times 1}^{{m}^{th} patient} ]}_{p \times m}$$

wherein p represents the number of principal components and m represents the number of patients in the datasets.

For the training of deep learning models which require global scatter plots as input variable, these global scatter plots are derived from the datasets according to the above-described methods:

$$[ {X}_{train} , {X}_{test} ] = {[ {[ Cluster plot ]}_{1}^{Patient} {… [ Cluster plot ]}_{m}^{Patient} ]}_{m}$$

wherein the datasets consist of m global scatter plots, wherein m represents the number of patients.

A true disease (positive) or a true healthy control case (negative) corresponds to each global vector and global scatter plot in the datasets. The truths are combined in a response vector or a one-hot encode response matrix Yₜᵣₐᵢₙ. In the case of one-hot encoding, categorical data are converted into numerical data for use during the machine learning. Categorical characteristics are hereby converted in particular into binary characteristics, which are “one-hot” encoded, i.e. if a characteristic is represented by this column it receives a 1, otherwise a 0.

The training of the models is effected on the training dataset according to the supervised and/or semi-supervised approach. Supervised learning is a subfield of machine learning. By learning is meant here the ability of artificial intelligence to reproduce regularities. The results are known through expert knowledge in the response matrix Yₜᵣₐᵢₙ and are used to teach the system. A learning algorithm attempts to find a hypothesis which makes predictions that are as accurate as possible. By hypothesis is meant here a mapping which maps the presumed output value to each input value (Rostamizadeh, Afshin., Talwalkar, Ameet.: Foundations of machine learning. MIT Press, Cambridge, MA 2012, ISBN 978-0-262-01825-8.). The method is therefore geared to an output that is defined beforehand and is to be learned, the results of which are known in the response matrix Yₜᵣₐᵢₙ. The results of the learning process can be compared with the known, correct results, thus “supervised” (Guido, Sarah, Rother, Kristian: Einführung in Machine Learning mit Python Praxiswissen Data Science [Introduction to Machine Learning with Python: A Guide for Data Scientists]. Heidelberg, ISBN 978-3-96009-049-6.).

Semi-supervised learning is an approach in machine learning in which, during the training, a small amount of data, the results of which are known in the response matrix Yₜᵣₐᵢₙ (labeled), is combined with a large amount of data, the results of which are unknown (unlabeled). In the first step, the first models are initially trained with the labeled data from the pre-built database. After that, the unlabeled data are classified with initially trained models and thereby labeled (pseudo-labeling). The database can be expanded with unlabeled data and corresponding pseudo-labels and used for the training of the next models. The pseudo-labels are here regarded as true diagnoses.

After the training of the models and their aggregation to form an ensemble, the quality of the ensembles is checked on the test dataset Xₜₑₛₜ. One of the possibilities for estimating the quality of the models is grouping the results in a confusion matrix.

Truth

| | | Negative | Positive |
|---|---|---|---|
| Prediction | Negative | TN | FN |
| | Positive | FP | TP |

wherein TN stands for true negative, FN for false negative, FP for false positive and TP for true positive predictions. The confusion matrix illustrates the number of patients with diseases or healthy patients, respectively, which were correctly or incorrectly predicted by models. The sensitivity here:

$$Sensitivity = \frac{TP}{TP + FN}$$

indicates the probability with which a positive diagnosis is correctly classified as positive. For example, in the case of a medical diagnosis, the sensitivity corresponds to the proportion of patients that were actually sick in which the disease was also detected. In addition to the sensitivity, the specificity here:

$$Specificity = \frac{TN}{TN + FP}$$

indicates the probability with which a ruling out of the disease is correctly classified as negative. For example, in the case of a medical diagnosis, the specificity corresponds to the proportion of healthy patients in which it was also determined that no disease was present. The specificity of a test indicates the probability with which a non-infected person would also actually be detected.

Within the framework of an experimental study, a database with 221 cases was retrospectively built in collaboration with the multidisciplinary hospital no. 2 in St. Petersburg. In 110 patients, the diagnosis was classified as acute coronary syndrome (55 cases with STEMI and 55 cases with NSTEMI) by a cardiologist. In 111 patients, the disease was ruled out; they thus represent the healthy control cases. In all patients, the first blood sample was taken in the emergency department and measured in a hematology analyzer of the CELL-DYN Sapphire type (Abbott Laboratories, USA). The database was divided at random into a training dataset Xₜᵣₐᵢₙ with 154 cases and a test dataset Xₜₑₛₜ with 67 cases. The training dataset Xₜᵣₐᵢₙ was used to train the machine and deep learning models. The accuracy and performance of the models was evaluated on the test dataset Xₜₑₛₜ. The test dataset Xₜₑₛₜ contained 17 STE-ACS, 18 NON-STE-ACS and 32 negative control cases. After the training of the models and their aggregation to form an ensemble, the quality of the ensembles was checked on the test dataset Xₜₑₛₜ. According to this, out of 32 healthy patients 30 were correctly classified as healthy (TN) and out of 35 sick patients 34 were correctly classified as sick (TP). This results in a sensitivity of 97.14% and a specificity of 93.75%, which indicates a very good quality of the ensembles. If the accuracies of the method described are compared with the accuracies of the High Sensitivity Troponin-I test from Abbott Technologies, such as is represented for instance in FIG. 11, it is thus found that the method described can deliver a better sensitivity and specificity immediately after the first blood examination.

Within the framework of the method, the trained models, which are trained on the basis of the training dataset and demonstrate a high accuracy on the test dataset, can be used within a laboratory information system for the prediction of diseases which affect the morphological characteristics and the cytoplasmic complexity of blood cells. The database can at any time be expanded with new labeled and/or unlabeled patient cases, with the result that the models can be trained again according to the supervised and/or semi-supervised approach, in order to obtain better models and to replace the old models where necessary.

All method steps of the computer-implemented method during the diagnosis of diseases that affect the morphological characteristics and the cytoplasmic complexity of blood cells are summarized in the example representation of FIG. 10.

The novelty of the solution presented is achieved in that, in comparison with existing analogs, for the first time global vectors and global scatter plots, which are obtained automatically from measured properties of leukocytes through the cluster analysis, are used as input variables for ensembles of machine and deep learning models, with the aim of diagnosing diseases that affect the morphological characteristics and the cytoplasmic complexity of blood cells. In the case of the diagnosis of NSTE-ACS, more accurate and faster results than modern conventional standard solutions were achieved immediately after the first blood sample within the framework of the experimental study, as shown by way of example in FIG. 11.

In an advantageous embodiment, obtaining the blood parameters of the full blood count comprises obtaining them from a hematological analyzer.

In an advantageous embodiment, reducing the dimension of the global vector comprises a principal component analysis, wherein the global vector is standardized before and/or after the dimension reduction.

In an advantageous embodiment, the at least one scatter plot image is standardized for the at least one deep learning model. This can preferably be effected before and/or during the transfer to the deep learning model in question.

In an advantageous embodiment, the ascertaining of the clusters comprises a cluster analysis, wherein the cluster analysis comprises a hierarchical cluster method.

In an advantageous embodiment, the diagnosis of at least one disease comprises the soft voting and/or hard voting method.

In an advantageous embodiment, the machine learning models which receive the reduced global vector as input variable in the ensemble comprise artificial neural networks, k-nearest neighbors, random forest, AdaBoost, gradient boosting machines (GBMs) and/or support vector machines (SVMs).

In an advantageous embodiment, the deep learning models which receive at least one scatter plot image as input variable comprise convolutional neural networks.

In an advantageous embodiment, all models in the ensemble are trained on the basis of a pre-built database, wherein the database comprises the measured properties of individual cells and/or scatter plots; wherein the database can be expanded with new measured properties of individual cells and/or scatter plots. The database could thus be constantly expanded with new data for the training of the models, in order to increase the accuracy of the models.

In an advantageous embodiment, the creation of at least one result report is effected by the computer, wherein the result report comprises a graphic and/or a text for information and/or a probability and/or a score for at least one disease, wherein the presentation of the result report comprises the presentation on the computing device and/or mobile device and/or laboratory device.

In an advantageous embodiment, the method furthermore comprises obtaining blood parameters from at least one patient, wherein the blood parameters comprise the measured properties of the individual cells of the full blood count, wherein the individual cells comprise erythrocytes and thrombocytes, wherein in b), in addition to or instead of at least one scatter plot, at least one histogram is created, wherein one of the axes of the histograms comprises the number of individual cells, wherein in f), in addition to or instead of at least one scatter plot image, at least one histogram image is used as input variable for at least one deep learning model in the ensemble for the diagnosis of at least one disease, wherein the database in claim 8 is supplemented with histograms.

The invention analogously relates to a system for diagnosing diseases that affect the morphological characteristics and the cytoplasmic complexity of blood cells, having a computer with a computing unit, a storage unit connected thereto and an input unit, wherein the system is formed to a) collect blood parameters from at least one patient obtained by an analyzer via the input unit, wherein the blood parameters comprise at least two measured properties of the individual cells of the full blood count, wherein the individual cells comprise the leukocytes, wherein the measured properties of the leukocytes comprise the size, granularity of the cytoplasm and the structure of the cell nucleus; b) create at least one scatter plot using the computing unit, wherein each axis of the scatter plot comprises a different measured property of the individual cells; c) ascertain at least one cluster in at least one of the at least one scatter plot using the computing unit, wherein the clusters comprise the subpopulations of the leukocytes, wherein the subpopulations comprise monocytes, lymphocytes, basophils, neutrophils and eosinophils; d) combine the property elements of the ascertained clusters to form a one-dimensional global vector using the computing unit, wherein the arrangement of the property elements in the global vector comprises an arrangement according to the associated clusters; e) reduce the dimension of the global vector using the computing unit; f) ascertain by means of the computing unit at least one disease using an ensemble, wherein the ensemble comprises at least one machine learning model and at least one deep learning model, wherein the machine learning model receives at least one reduced global vector as input variable and the at least one deep learning model receives at least one scatter plot image as input variable; and g) automatic generation of a report which comprises at least one result of the diagnosis of at least one disease using the computing unit.

The system could furthermore be formed to perform one or more of the method steps described previously. It is to be noted here that the storage unit, which is connected to the computing unit, is formed to store a corresponding algorithm, to supply it to the computing unit for execution, to store data and to supply it to the computing unit during execution of the algorithm or the method. The input unit can be any desired apparatus which is capable of transferring data from outside to the system. This can be a data or signal interface, an input device, a data carrier with a corresponding interface or any other suitable apparatus.

### BRIEF DESCRIPTION OF THE DRAWINGS

FIG. 1 shows what properties of the leukocytes are measured with a laser light in the hematological analyzer (Abbott Cell Dyn Sapphire): size, lobularity, granularity and complexity. This is an example of the analyzer of the Abbott Cell Dyn Sapphire type. In other analyzers, for example of the Sysmex type, cell size, cell complexity and cell contents can be measured. As noted previously, suitable analyzers can usually measure at least the two properties: cell size and internal complexity.

FIG. 2 shows by way of example a series of subpopulations of the leukocytes, which are sorted by measured properties through the cluster analysis, wherein the subpopulations comprise basophils, neutrophils, lymphocytes, monocytes and eosinophils. All subpopulations are described by their properties.

FIG. 3 shows by way of example the ascertaining of at least three subpopulations (neutrophils, lymphocytes, monocytes) according to at least three properties (size, complexity, lobularity) by plotting the measured properties against each other in at least two scatter plots in Cartesian coordinates: size against complexity and size against lobularity.

FIG. 4 shows by way of example the standardization and reduction of the global vector which is used as input variable for the machine learning models.

FIG. 5 shows by way of example a global scatter plot which is used as input image in the form of a matrix for the deep learning models.

FIG. 6 shows by way of example the creation of a global scatter plot by plotting the measured properties of the individual cells of the leukocytes against each other in individual scatter plots: size against complexity, size against granularity, size against lobularity, complexity against granularity, complexity against lobularity, granularity against lobularity, wherein the order of the individual scatter plots in the global scatter plot can be changed.

FIG. 7 shows by way of example the above-named hard voting method for the diagnosis of the disease.

FIG. 8 shows by way of example the likewise previously named soft voting method for the diagnosis of the disease.

FIG. 9 is a schematic diagram illustrating a use of a database for training and evaluating of models.

FIG. 10 is a schematic diagram summarizing all steps of the computer-implemented method.

FIG. 11 is a chart comparing accuracies of the method described herein with accuracies of a High Sensitivity Troponin-I test from Abbott Technologies

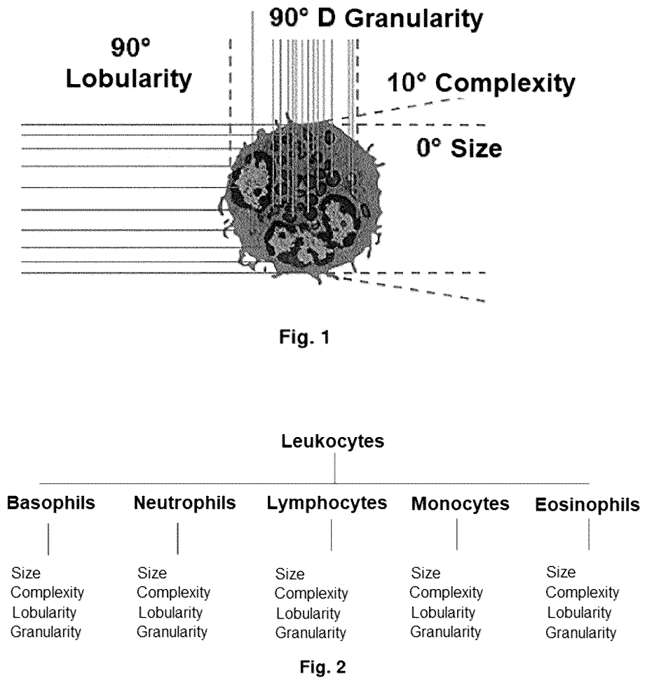

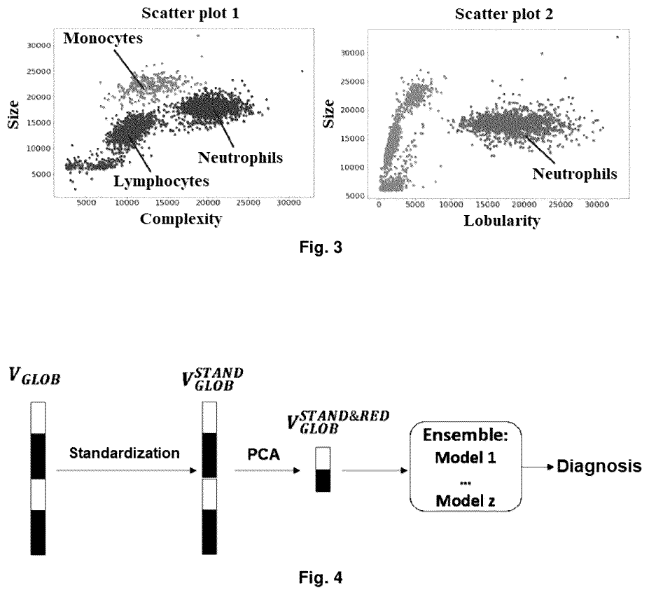

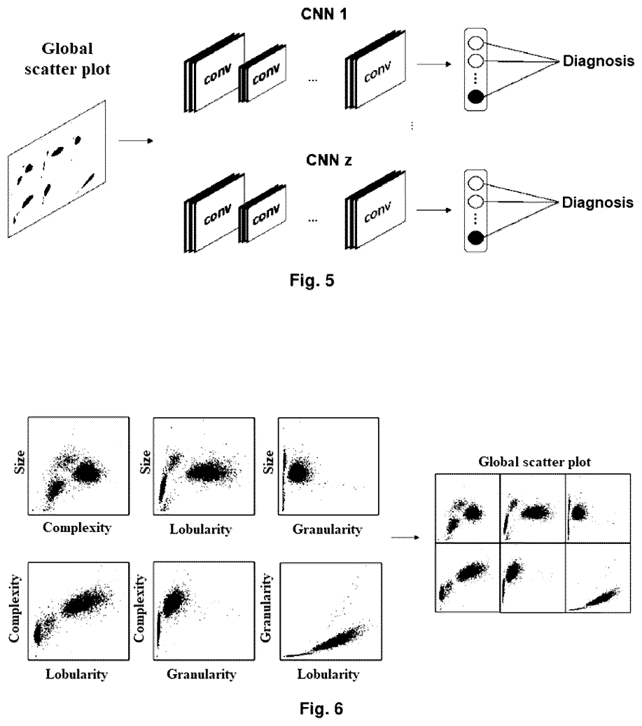

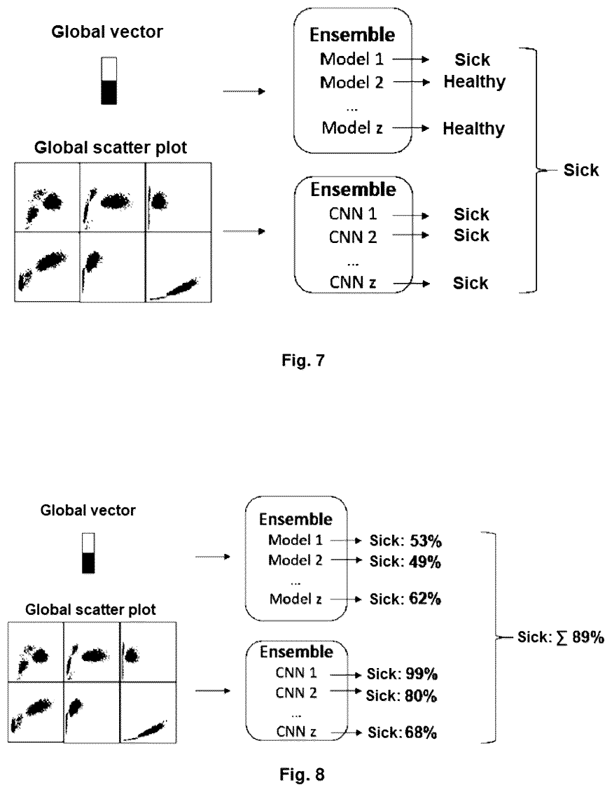

### DETAILED DESCRIPTION OF THE INVENTION

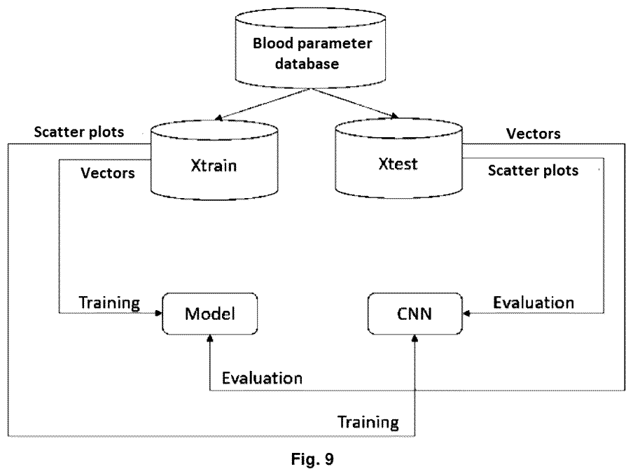

The use of a database for the training and evaluating of the models is shown by way of example in FIG. 9. The database contains the measured blood parameters and the corresponding true diagnoses (labeled database). The database is divided into two parts: Xₜᵣₐᵢₙ and Xₜₑₛₜ. Xₜᵣₐᵢₙ is used for the training, whereas Xₜₑₛₜ is used for the evaluating of the models. For the models which require the global vectors as input, global vectors are obtained from Xₜᵣₐᵢₙ and Xₜₑₛₜ according to above-described approaches. For the models which require the global scatter plots as input, global scatter plots are obtained according to above-described approaches.

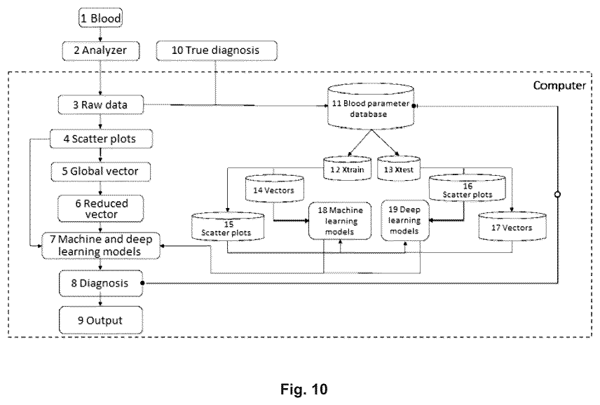

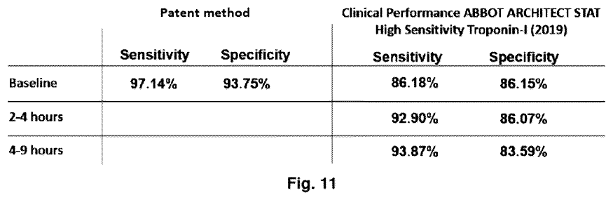

All steps of the computer-implemented method are summarized in the example representation of FIG. 10. A blood sample taken from a patient before the method is provided for examination in the method according to the invention, which is denoted as step (1) here. In step (2), the properties of the individual cells of the blood sample are measured in an analyzer. In step (3), the measured data are transferred as raw data to the computer. In step (4), the scatter plots are created. In step (5), the subpopulations are ascertained, differentiated by their properties, through the cluster analysis and combined in a global vector. In step (6), the global vector is standardized and reduced. In step (7), the reduced vector is used as input variable for the at least one trained machine learning model, while the individual scatter plots from (4) are combined in a global scatter plot and used as input image for the at least one trained deep learning model. In step (8), the diagnosis of the disease is effected according to the hard or soft voting approach. In step (9), the diagnosis is output on a graphical interface or in the form of an automatically created report. In step (11), the predicted diagnosis can be stored together with corresponding raw data from (3) in the pre-built database, in order to expand the database with a new case and to train new models according to the semi-supervised approach. The pre-built database can also be expanded with raw measured data and the corresponding true diagnosis in step (11). The purpose of the database is to train and evaluate the models. The database is divided into the training dataset and test dataset in steps (12) and (13). In step (18), the training and evaluating of the at least one machine learning model is effected, wherein global vectors from steps (14) and (17) are used as input variables. In step (19), the training and evaluating of the at least one deep learning model is effected, wherein scatter plots from steps (15) and (16) are used as input variables. The models in step (7) can be replaced with the newly trained models in steps (18) and (19) at any time, as long as the newly trained models on the test dataset achieve better results.

The described method can be carried out on the basis of the clinical blood analysis results which are obtained during the first minutes of the patient's stay in the emergency department even before receipt of other laboratory and instrumental examination methods, which improves the quality of the medical care considerably.

An example of the application of the method in the case of a patient with a “positive” diagnosis, which was confirmed as a result of the application of the method.

The following example is to serve for the further explanation of the method according to the invention. A 36-year-old patient entered the admission ward with a preliminary diagnosis of “arteriosclerotic heart disease, acute coronary syndrome without ST elevation. Acute cardiac insufficiency. Killip class I”, two hours after a typical pain syndrome. The electrocardiographic examination was carried out in the receiving section and likewise showed no elevation of the ST segment on the electrocardiogram. Venous blood samples were then taken for laboratory tests. The blood samples taken outside the method were provided for examination (FIG. 10, step 1), wherein the examination also comprised the high sensitivity cardiac troponin-I method and clinical blood analysis. The results of laboratory studies were as follows: urea 4.2 mmol/l (3.0-9.2); ALT 16 units/I (0-55); AST 12 units/I (5-34); total protein 70 g/l (64-83); creatinine 74 μmol/l (64-111); total bilirubin 6.2 μmol/l (3.4-20). 5); glucose 7.5 mmol/l (3.9-5.5); potassium 3.7 mmol/l (3.5-5.1); sodium 137 mmol/l (135-145); ionized calcium 1.23 mmol/l (1.13-1.32); APTV 78.7 s (25.1-36.5); MNO 0.97 (0.2-0.5). 90-1.20); prothrombin 118.0% (70.0-140.0); prothrombin time 11.0 s (9.4-12.5); leukocytes 12.4 10E9/I (4.0-9.0); (NEUT) neutrophils 10.0*109/1 (2.0-5.5); (NEUT %) neutrophils 80.0% (48.0-78.0); (LYM) lymphocytes 1.79*109/1 (1.20-3.00); (LYM %) lymphocytes 14.3% (19.0-37.0); (MON) monocytes 0.57 109/1 (0.09-0.60); (MON %) monocytes 4.6% (3.0-11.0); (EOS) eosinophils 0.07*109/I (0.00-0.30); (EOS %) eosinophils 0.52% (1.00-5.00); (BAS) basophils 0.07*109/1 (0.00-0.06); (BAS %) basophils 0.52% (0.00-1.00); (HGB) hemoglobin 134 g/l (130-160); (HCT) hematocrit 40.7% (40.0-48). 0); (RBC) erythrocytes 4.45 10*12/1 (4.00-5.60); (MCH) mean corpuscular hemoglobin concentration 30.1 pg (24.0-34.0); (MCHC) mean corpuscular hemoglobin concentration 32.9 g/dL (30.0-38.0); (MCV) mean corpuscular volume 91.4 fL (75.0-95.0); (RDW-CV) red blood cell distribution width about 11.0% (11.5-16.0%). 5); (PLT) platelets 262*109/I (180-400); (MPV) mean platelet volume 11.5 fL (7.4-10.4); cardiac troponin I (high sensitivity method) 37.9 ng/ml (upper reference limit 26.2 ng/ml).

After the blood was measured (FIG. 10, step 2), the raw measured data were transferred to the PC (FIG. 10, step 3). Then, two scatter plots were produced for the trained deep learning models (FIG. 10, step 4) and combined to form a global scatter plot. In parallel, the global vector was derived with 4216 elements (FIG. 10, step 5):

$${V}_{GLOB} = {( \begin{matrix}\{ {V}_{Neutrophils} \} \\ \{ {V}_{Lymphocytes} \} \\ \{ {V}_{Monocytes} \}\end{matrix} )}_{4216}$$

wherein 4216 elements represent the measured properties of the subpopulations of the leukocytes. The global vector is standardized by removing the mean and scaling to a single dispersion on the basis of a predefined database:

$${V}_{GLOB}^{STAND} = \frac{\{ {V}_{GLOB} \} - {\{ u \}}_{Database}}{{\{ s \}}_{Database}}$$

wherein the vector {u} represents the means of the property elements of the global vectors in the pre-built database and the vector {s} represents the standard deviations of the elements of the global vectors in the pre-built database.

After the standardization, the PCA was applied (FIG. 10, step 5) in order to reduce the dimensionality of characteristics of 4216 elements to 4 elements, which are called principal components, and at the same time to obtain the greatest possible variability (information) of the characteristics. After the application of the principal component analysis, all 4216 elements of the standardized global vector were reduced to the vector in the 4-dimensional subspace:

$${V}_{GLOB}^{STAND \& RED} = \{ \begin{matrix}- 40.48 \\ 58.66 \\ - 5.4 \\ 0.75\end{matrix} \}$$

At first glance, it is difficult to extract information for the patient diagnosis from the values of this reduced vector. For this purpose, an ensemble consisting of the ensembles of the trained machine learning models is used. The ensemble consists of an ensemble of artificial neural networks, an ensemble of k-nearest neighbors models, an ensemble of random forest models, an ensemble of AdaBoost models, an ensemble of gradient tree boosting models and an ensemble of support vector machine models, wherein the individual ensembles were each trained on the pre-built database. The standardized and reduced global vector is used as input vector for all ensembles, whereas the global scatter plot is used for a deep learning model. For the above-named case, the votes for ACS of individual ensembles are counted (hard voting):

| | Ensemble of | Positive (ACS) | Negative (no ACS) |
|---|---|---|---|
| 1 | k-nearest neighbors | YES | No |
| 2 | neural networks | YES | No |
| 3 | random forest | YES | No |
| 4 | gradient boosting | YES | No |
| 5 | AdaBoost | YES | No |
| 6 | support vector machines | YES | No |
| 7 | deep learning CNN | YES | No |

The end result for ACS after the hard voting method is positive. The decision was taken to carry out a percutaneous coronary intervention. The patient had a coronary angiographic examination, followed by a transluminal dilatation and stenting of the coronary artery subject to infarction.

CORONARY ANGIOGRAPHY No. 7175 dated 7 Jun. 2018: blood circulation on the left side. Left coronary artery: barrel—without stenosis. Anterior ventricular branch—stenosis of the opening 5-50%, subocclusion in the middle third. Ramus intermedius—stenosis in the proximal third of 90%. Diagonal branches—without stenosis. Bending branch (BB)—without stenosis. Obtuse marginal branches—without stenoses. Right coronary artery: hypoplastic. Acute marginal branch: no stenosis. Rear branch (ROB)—without stenoses. Posterior interventricular branch—without stenoses.

CORONARY ANGIOPLASTY AND PMV STENTING No. 7176 dated Jun. 7, 2018: Stenosis area of the PMV (average third) BC 2.0*20.0 mm, p=18 atm. The stent with a medicament coating of 2.75*33.0 mm is implanted in the middle third of the BC 2.0*20.0 mm, p=16 atm. Control: TIMI grade III blood flow. No infiltration shadows were found on chest X-ray images in 2 projections dated 8 Jun. 2018. The roots are structurally not enlarged; the left one is partially blocked. The lung pattern was not altered. The diaphragm is defined. Cardiac shadow without features. Sinus is clear.

The following treatment was carried out: beta blockers, anticoagulants, double disaggregation therapy, statins (the dose of Crestor was reduced by 20->10 mg/day due to the increase in the transaminase level), gastroprotectants. The patient declined a rehabilitation treatment in a sanatorium.

In the postoperative phase, the maximum concentration of cardiac troponin I in dynamic observation reached 7522.5 ng/ml. The stay in hospital lasted 12 days. The final diagnosis was “arteriosclerotic heart disease. Acute myocardial infarction of the anterior parietal region, high lateral portions of the left ventricle without ST segment elevation dated Jun. 7, 2018. Coronary angioplasty and stenting dated Jun. 7, 2018”. The patient was discharged on Jun. 19, 2018 for further out-patient observation at home.

1. A computer-implemented method for diagnosing diseases that affect the morphological characteristics and the cytoplasmic complexity of blood cells, wherein the method comprises:
a) obtaining blood parameters from at least one patient, wherein the blood parameters comprise at least two measured properties of the individual cells of the full blood count, wherein the individual cells comprise the leukocytes, wherein the measured properties of the leukocytes comprise the size, granularity of the cytoplasm and the structure of the cell nucleus;
b) creating at least one scatter plot, wherein each axis of the scatter plot comprises a different measured property of the individual cells;
c) ascertaining at least one cluster in at least one of the at least one scatter plot, wherein the clusters comprise the subpopulations of the leukocytes, wherein the subpopulations comprise monocytes, lymphocytes, basophils, neutrophils and eosinophils;
d) combining the property elements of the ascertained clusters to form a one-dimensional global vector, wherein the arrangement of the property elements in the global vector comprises an arrangement according to the associated clusters;
e) reducing the dimension of the global vector;
f) diagnosing at least one disease using an ensemble, wherein the ensemble comprises at least one machine learning model and at least one deep learning model, wherein the machine learning model receives at least one reduced global vector as input variable and the at least one deep learning model receives at least one scatter plot image as input variable; and
g) automatically generating a report which comprises at least one result of the diagnosis of at least one disease.

2. The computer-implemented method according to claim 1, wherein obtaining the blood parameters of the full blood count comprises obtaining them from a hematological analyzer.

3. The computer-implemented method according to claim 1, wherein reducing the dimension of the global vector comprises a principal component analysis, wherein the global vector is standardized before and/or after the dimension reduction.

4. The computer-implemented method according to claim 1, wherein the at least one scatter plot image is standardized for the at least one deep learning model.

5. The computer-implemented method according to claim 1, wherein the ascertaining of the clusters comprises a cluster analysis, wherein the cluster analysis comprises a hierarchical cluster method.

6. The computer-implemented method according to claim 1, wherein the diagnosis of at least one disease comprises the soft voting and/or hard voting method.

7. The computer-implemented method according to claim 1, wherein the machine learning models which receive the reduced global vector as input variable in the ensemble comprise artificial neural networks, k-nearest neighbors, random forest, AdaBoost, gradient boosting machines (GBMs) and support vector machines (SVMs).

8. The computer-implemented method according to claim 1, wherein the deep learning models which receive at least one scatter plot image as input variable comprise convolutional neural networks.

9. The computer-implemented method according to claim 1, wherein all models in the ensemble are trained on the basis of a pre-built database,
wherein the database comprises the measured properties of individual cells and/or scatter plots; wherein the database can be expanded with new measured properties of individual cells and/or scatter plots.

10. The computer-implemented method according to claim 1, wherein the creation of at least one result report is effected by the computer, wherein the result report comprises a graphic and/or a text for information and/or a probability and/or a score for at least one disease, wherein the presentation of the result report comprises the presentation on the computing device and/or mobile device and/or laboratory device.

11. The computer-implemented method according to claim 1, furthermore comprising: obtaining blood parameters from at least one patient, wherein the blood parameters comprise the measured properties of the individual cells of the full blood count, wherein the individual cells comprise erythrocytes and thrombocytes, wherein in 1b), in addition to or instead of at least one scatter plot, at least one histogram is created, wherein one of the axes of the histograms comprises the number of individual cells, wherein in 1f), in addition to or instead of at least one scatter plot image, at least one histogram image is used as input variable for at least one deep learning model in the ensemble for the diagnosis of at least one disease, wherein the database in claim 9 is supplemented with histograms.

12. A system for diagnosing diseases that affect the morphological characteristics and the cytoplasmic complexity of blood cells, having a computer with a computing unit, a storage unit connected thereto and an input unit, wherein the system is formed to
a) collect blood parameters from at least one patient obtained by an analyzer via the input unit, wherein the blood parameters comprise at least two measured properties of the individual cells of the full blood count, wherein the individual cells comprise the leukocytes, wherein the measured properties of the leukocytes comprise the size, granularity of the cytoplasm and the structure of the cell nucleus;
b) create at least one scatter plot using the computing unit, wherein each axis of the scatter plot comprises a different measured property of the individual cells;
c) ascertain at least one cluster in at least one of the at least one scatter plot using the computing unit, wherein the clusters comprise the subpopulations of the leukocytes, wherein the subpopulations comprise monocytes, lymphocytes, basophils, neutrophils and eosinophils;
d) combine the property elements of the ascertained clusters to form a one-dimensional global vector using the computing unit, wherein the arrangement of the property elements in the global vector comprises an arrangement according to the associated clusters;
e) reduce the dimension of the global vector using the computing unit;
f) ascertain by means of the computing unit at least one disease using an ensemble, wherein the ensemble comprises at least one machine learning model and at least one deep learning model, wherein the machine learning model receives at least one reduced global vector as input variable and the at least one deep learning model receives at least one scatter plot image as input variable; and
automatic generation of a report which comprises at least one result of the diagnosis of at least one disease using the computing unit.
